(19) United States
(12) Patent Application Publication
(10) Pub. No.: US 2012/0265168 A1
(43) Pub. Date: Oct. 18, 2012

(54) TUMESCENT ANESTHESIA DELIVERY IN CONNECTION WITH ENDOVENOUS VEIN THERAPY

(76) Inventors: John D. Horowitz, Windermere, FL (US); Douglas E. Marcum, Oviedo, FL (US)

(21) Appl. No.: 13/390,179

(22) PCT Filed: Aug. 12, 2010
(86) PCT No.: PCT/US10/45282

§ 371 Date: Jul. 5, 2012

Related U.S. Application Data
(60) Provisional application No. 61/233,211, filed on Aug. 12, 2009.

(51) Int. Cl. A61M 19/00

(52) U.S. Cl. 604/512; 604/272

(57) ABSTRACT

Disclosed herein are methods for delivering a tumescent anesthetic fluid to an area surrounding a target vein by inserting a needle into a location proximate to the target vein. The needle includes a proximal and a distal end, a pointed needle tip an elongated shaft body with a hollow center which extends throughout its length. The shaft body is of a length sufficient to penetrate epidermal, dermal and subdermal layers of a patient's lower extremity, and multiple openings are positioned along the length of the shaft body for administering the anesthetic through into said the surrounding area. The administration of tumescent anesthesia allows fewer needle pricks while simultaneously providing greater distribution of the anesthetic to the target area of the patient.

### RELATED APPLICATIONS

This application claims priority to U.S. Ser. No. 61/233,211 filed Aug. 12, 2009, which is incorporated herein in its entirety.

### FIELD OF THE INVENTION

This invention relates to the administration of tumescent anesthesia, and more particularly to a device and a method for a more efficient delivery of tumescent anesthesia to an area surrounding a target vein in a patient.

### BACKGROUND OF THE INVENTION

Veins can be broadly divided into three categories: the deep veins, which are the primary conduit for blood return to the heart; the superficial veins, which parallel the deep veins and function as a channel for blood passing from superficial structures to the deep system; and topical or cutaneous veins, which carry blood from the skin or subcutaneous tissue, or in some instances musculature to the superficial system. Veins are thin-walled and contain one-way valves that control blood flow. Normally, the valves open to allow blood to flow into the deep veins and close to prevent back-flow into the superficial veins. When the valves are malfunctioning or only partially functioning, however, they no longer prevent the back-flow of blood into the superficial veins. This condition is called venous reflux. As a result of reflux, venous pressure builds within the superficial system. This pressure is transmitted to topical (superficial) veins, which, because the veins are thin walled and not able to withstand the increased pressure, become dilated, tortuous or engorged. These superficial, engorged veins lose their useful purpose of transporting venous blood back to the heart and become a reservoir for blood become stagnant and to pool in the lower extremities. This stagnant blood leads to elevated venous pressure, or venous hypertension, and predisposes to phlebitis, venous thrombosis, soft tissue scarring, ulceration and also the symptoms of pain, swelling, and fatigue that accompanies venous insufficiency.

In particular, venous reflux in the lower extremities is one of the most common medical conditions of the adult population. It is estimated that venous reflux disease affects approximately 25% of adult females and 10% of males. Symptoms of reflux include painful varicose veins, heaviness, tiredness, swelling, itching burning and cosmetically unsightly veins. If left untreated, venous reflux may cause severe medical complications such as bleeding, phlebitis, ulcerations, thrombi and lipodermatosclerosis.

Endovenous thermal therapy is a relatively new treatment technique for venous reflux diseases. With this technique, thermal energy generated by laser or radiofrequency energy is delivered to the inner vein wall causing vessel ablation or occlusion. Typically a catheter, fiber or other delivery system is percutaneously inserted into the lumen of the diseased vein under ultrasound guidance. Thermal energy is delivered from the distal end of the delivery system as the device is slowly withdrawn through the vein. Although the device description described herein focuses on endovenous treatment using laser energy, other thermal energy forms may be used.

According to a typical case, using the main superficial vein as an example, the procedure begins with an introducer sheath being placed into the main superficial vein, also called the great saphenous vein, at a distal location and advanced to within a few centimeters of the point at which the great saphenous vein enters the deep vein system, (the sapheno-femoral junction). Typically, a physician will measure the distance from the insertion or access site to the sapheno-femoral junction on the surface of the patient's skin. This measurement is then transferred to the sheath using tape, a marker or some other visual indicator to identify the insertion distance on the sheath shaft. Other superficial veins may be accessed depending on the origin of reflux.

It is to be understood that the great saphenous vein is not the only vein treated by the present therapy. Other veins include, but are not limited to, the lesser saphenous, branch veins, or perforator veins. In essence, any vein of the superficial system that will permit passage of the fiber can be treated according to the techniques discussed herein.

The sheath is placed typically with the assistance of ultrasound guidance. The physician inserts the sheath into the vein using the visual mark on the sheath as an approximate insertion distance indicator. Ultrasound is then used to guide final placement of the tip relative to the junction. Positioning of the sheath tip relative to the sapheno-femoral junction or other reflux point is critical to the procedure because the sheath tip position is used to confirm correct positioning of the fiber when it is inserted and advanced. Conventionally used sheath tips are often difficult to clearly visualize under ultrasound guidance.

Prior to the application of thermal energy, tumescent anesthesia is injected along the entire length of the vein into space between the vein and the surrounding perivenous tissue. A mixture of saline, bicarbonate and 0.1-0.5% lidocaine or other similar anesthetic agent is typically used. Tumescent anesthesia serves several functions. The fluid anatomically isolates the vein, creating a barrier to protect the tissue and nerves from the thermal energy. Specifically, the fluid provides a heat sink to prevent thermal injury to adjacent non-target tissues, nerves and the skin surface. Extrinsic pressure from the fluid also compresses the vessel, reducing the vein diameter, minimizing the volume of the vein, and maximizing the heat affect to the vein walls. Finally, the lidocaine mixture, with its anesthetic characteristics, reduces patient pain during the procedure.

The tumescent injections are typically administered every few centimeters along the entire length of the vein under ultrasonic guidance. Ultrasound is used to visualize the vein, confirm proper location of the needle tip in the perivenous space, and to determine correct injection volumes. After the user has confirmed that the needle tip is correctly positioned between the vein and perivenous tissue through ultrasonic imaging, the tumescent fluid is rapidly and forcefully injected. Again, visualization of the target perivenous space is often difficult, or even more commonly, the needle tip as it approaches the perivenous space is difficult to visualize. The inventors have now realized that this increases the chances that a user may inadvertently puncture the sheath wall or laser fiber with the needle tip during placement. In addition to the potential damage that could occur by incorrect needle tip placement, misplacement of the needle tip dramatically decreases the efficiency and/or accuracy of the anesthesia delivery.

Once the combined sheath/optical fiber assembly is properly positioned and after the administration of tumescent anesthesia as described above, thermal energy can be applied to the vein. To treat the vein, a laser generator is activated causing energy to be emitted from the distal end of the optical fiber into the vessel. The energy reacts with the blood remaining in the vessel and causes heat, which damages the vein wall which, in turn, causes cell necrosis and eventual vein collapse ablation by coaptation. With the energy source turned on, the sheath and fiber are slowly withdrawn as a single unit until the entire diseased segment of the vessel has been treated.

### BRIEF DESCRIPTION OF THE DRAWINGS

A more particular description of the invention briefly described above will be rendered by reference to specific embodiments thereof that are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1a-c depicts a transverse cross-sectional view of an embodiment of a method of injecting a needle for the delivery of tumescent anesthesia to an area surrounding a target vein.

FIG. 2a-c depicts a longitudinal cross-sectional view of the method shown in FIGS. 1a-c.

FIG. 3 depicts a cross-sectional view of an exemplary method embodiment.

FIG. 4 depicts a side view of an embodiment of a needle with multiple openings.

FIG. 5 depicts a side view of an embodiment of a needle with multiple openings.

FIG. 6 depicts a side view of an embodiment of a needle with multiple reflective scores surrounding each opening.

FIG. 7 depicts a side view of a needle embodiment with progressively smaller openings.

FIG. 8 depicts a cross-sectional side view of FIG. 9 (along the axis of the arrows) showing a reflective score surrounding an opening in the needle wall.

FIG. 9 shows a side view of the distal portion of a needle, with reflective scores surrounding openings in the needle wall, and arrows to demonstrate the axis upon which the cross section in FIG. 8 is shown.

FIG. 10 shows a more oblique angled approach, demonstrating the needle providing a broad area of delivery of tumescent anesthetic fluid to the area surrounding a target vein via a single needle prick.

### DETAILED DESCRIPTION OF THE INVENTION

The inventors have realized that the application of anesthesia in conjunction with endovenous treatment needs improvement. Embodiments of the invention are based on the inventors' development of a needle device that includes modifications that result in more efficient delivery of anesthesia (e.g. tumescent anesthesia). The needle device and methods of using the same decreases the amount of time required for administering anesthesia and decreases the amount of needle pricks required for such administration. Furthermore, the inventors have developed a needle which allows visualization during the procedure thus resulting in a more controlled, safer and more precise placement of the needle in the target area.

In one embodiment, the invention pertains to a method of delivering a tumescent anesthetic fluid to an area surrounding a target vein, by inserting a needle into a location proximate to the target vein. The needle is comprised of a proximal end and a distal end, a pointed needle tip on the distal end, an elongated shaft body with a hollow center which extends throughout its entire length. The shaft body is a length sufficient to penetrate the epidermal, dermal and subdermal layers of a patient's lower extremity. The needle also includes multiple openings positioned along the length of the shaft body for administering the anesthetic through the openings into the area surrounding the target vein. The embodiment allows administration of the tumescent anesthetic fluid to the location such that it ejects through the multiple openings simultaneously into the area surrounding the target vein.

In a more specific embodiment, the needle may also contain two or more reflective scores positioned along the length of the shaft. In another embodiment, the scores are contiguous with the openings located along the shaft of the needle operating as a guide to allow a user to correctly position the needle with ultrasonic or fluoroscopic imaging into the target area of the patient.

In yet another embodiment, the openings along the shaft body vary in diameter, wherein at least one opening has a different diameter than another opening. In another embodiment, the opening at the tip of the needle has a smaller diameter than the openings along the shaft body. The variations in diameter of the openings along the shaft of the needle and at the tip of the needle change the pressure of the fluid inside the needle while in use so that the fluid can more easily exit through the openings as it travels down the length of the shaft toward the distal end of the needle.

Another embodiment of the present invention provides a needle for injection of an anesthetic fluid. The needle has a proximal end and a distal end, a pointed tip and a hollow shaft, multiple reflective scores and multiple openings positioned along the shaft. In a more specific embodiment, the needle contains openings and scores contiguous with one another along the length of the shaft. In yet another embodiment, the needle contains reflective scores which surround each opening.

In yet a more specific embodiment, the invention relates to a needle containing multiple openings positioned along the shaft beginning at a location 0.1 cm-5 cm from the distal end and ending at a location about 0.5 cm-5 cm from the distal end, wherein each opening is optionally spaced about 0.01-1 cm apart, and each opening is optionally 0.01-0.5 centimeters in diameter. In a further embodiment, the openings are spaced equidistance apart.

In another embodiment, the invention pertains to a kit with a plurality of needles of varying lengths used for tumescent anesthesia for injection in the area peripheral to a venous body in a lower extremity of a patient. The kit contains needles varying in length for easier use with patients of varying size. The needles are comprised of a shaft, a proximal end, a distal end, and a pointed tip at the distal end. The shaft is comprised of multiple openings positioned along its length for flushing tumescent anesthesia through the openings, while positioned in the area peripheral to a venous body in said patients' lower extremity. This allows for greater spread of the fluid around the target vein of the patient with fewer needle pricks, and increased contact of the fluid with the perivenous(sp) area. The shaft also contains multiple scores for identification and localization of the needle during use with ultrasonic imaging, wherein the scores are contiguous with the openings along the shaft.

In a further embodiment, the openings of the needles are spaced between the scores along the shaft. In yet another embodiment, the shaft ranges in size from 3 cm-7 cm. Different sized shafts also comprise varying distances from the distal end that the openings begin. These variations in shaft length, and spacing of the scores and openings along the shaft, account for patients of varying sizes.

In yet a further embodiment, the invention is directed to a needle for injecting an area surrounding a target vein in a lower extremity of a patient containing a proximal end and a distal end, a hollow shaft connecting both ends, the distal end including a pointed tip, and the pointed tip including a hole. In specific embodiments, the needle length varies from 3-10 centimeters between the proximal and distal ends, the shaft contains a plurality of openings positioned along its length beginning at a location 0.1 cm-1.5 cm from the distal end and ending at a location 0.5 cm-5 cm from the distal end. Each opening is optionally spaced about 0.01-1 cm apart, and each opening is optionally 0.01-0.5 centimeters in diameter. In an alternative embodiment, the shaft contains reflective scores positioned along its length. In another embodiment, reflective scores are contiguous with each opening along the length of the shaft.

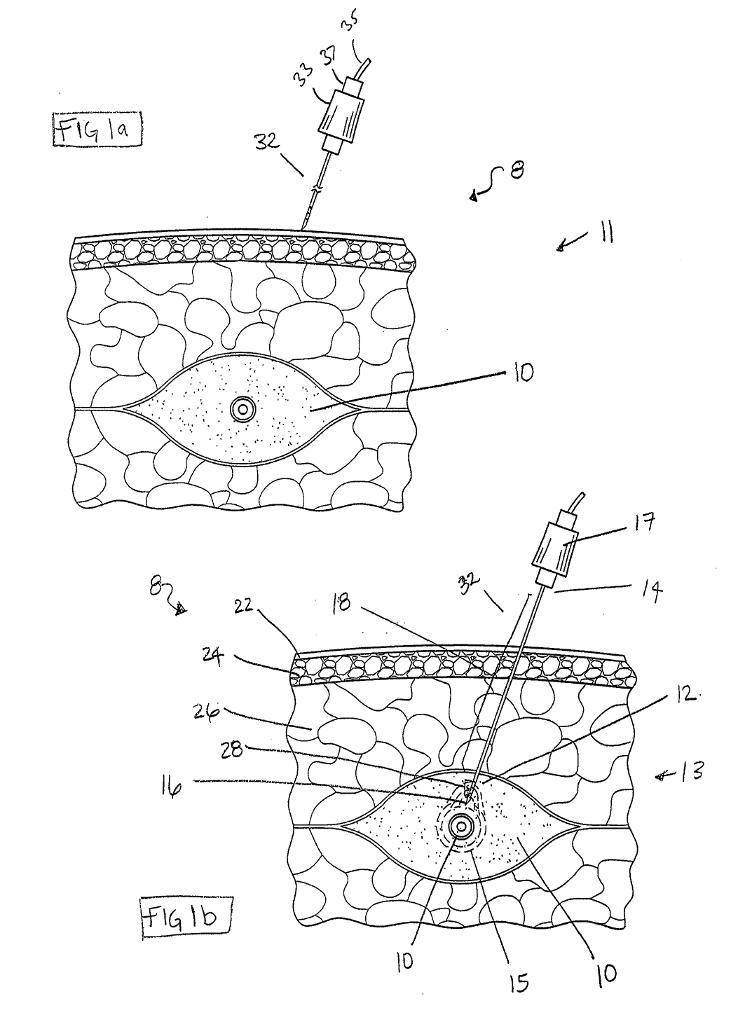

Turning to the drawings, FIG. 1 a-c illustrates an exemplary embodiment of a method 8 for the delivery of a tumescent anesthetic fluid to an area surrounding a target vein 10. In a first step 11, illustrated in FIG. 1a, a needle 32 is inserted into a location proximate to the target vein 10. In a second step 13, illustrated in FIG. 1b, the tumescent anesthesia fluid 15 is administered to the perivenous area 10. The needle 32 can be described as containing a proximal end 14, an end piece 17 on the proximal end which could be in the form of a LuerLoc for engaging to a syringe. The needle 32 also contains a distal end 12, a pointed needle tip 16 on the distal end 12, an elongated shaft body 18 with a hollow center 20 which extends throughout the entire length of the shaft body 18. The shaft body 18 is of a length sufficient as to penetrate the epidermal 22, dermal 24 and subdermal 26 layers of the skin in the lower extremity of a patient. Multiple openings 28 are positioned along the length of the shaft body 18 to allow for the administration of anesthetic through the openings 28 into the area surrounding the target vein 10. The tumescent anesthetic fluid is administered to the location such that it is ejected through the multiple openings 28 simultaneously into the area surrounding the target vein 10. As used herein, the term ‘simultaneous’ or ‘simultaneously’ when used to describe the delivery of fluid through the openings in the needle means that at some point in time, fluid is being extruded through all of the openings. The method 8 sets forth a unique and efficient manner for the effective delivery of tumescent anesthesia to the area surrounding a vein 10 in various types of patients. FIG. 1c shows how the tumescent anesthesia fluid 15 surrounds the target vein 30.

FIGS. 2a and 2b illustrate a cross-section of the method revealed in FIGS. 1a-c, further demonstrating how the vein 30 contracts during the thermal energy procedure. As is shown in the figures, the vein closes upon application of thermal energy by the probe 9. The tumescent anesthetic fluid 15 protects the surrounding tissue 10.

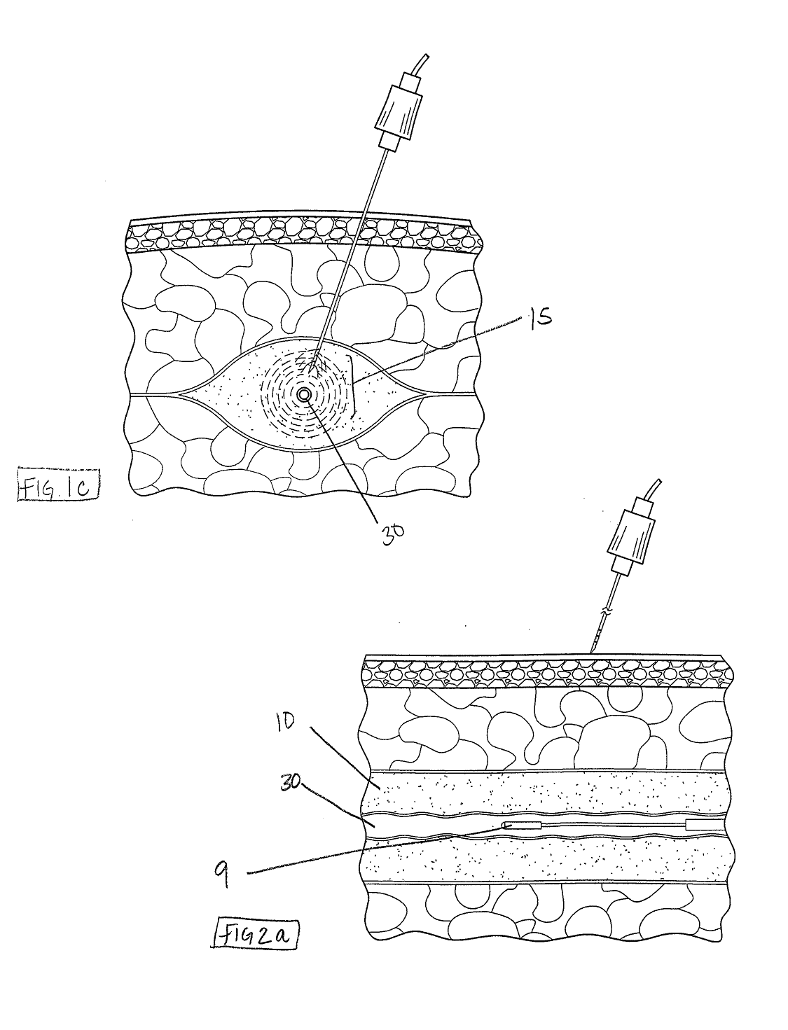

FIG. 3 depicts the effect of multiple openings 28 in the needle 32, even though the needle is to the side of the probe 9. This function allows the user room for error and is an advantage as it provides a surgeon with additional target area for the effective delivery of the tumescent anesthetic fluid 15, as can be seen in FIG. 3 where the needle 32 is located adjacent to the target vein 30 and is still effective.

FIG. 4 shows a side view of an embodiment of a needle 32, used for injection of an anesthetic fluid. The needle 32 includes a proximal end 14, a distal end 12, a pointed tip 16, and a hollow 20 shaft body 18 extending from proximal end to distal end to allow for the passage of fluid through the shaft 18 of the needle 32. The needle 32 also contains multiple reflective scores 34 and multiple openings 28 positioned along the shaft 18. The reflective scores 34 allow the needle 32 to be identified easily while inside the body of the patient. In another embodiment, the needle 32 contains multiple openings 28 and scores 34 contiguous with one another along the length of the shaft 18. D1 represents an area in this particular embodiment in which there are features of the needle whether it be openings 28 or reflective scores 34. D2 represents the area, in this embodiment, in which the reflective scores 34 are located on the needle 32.

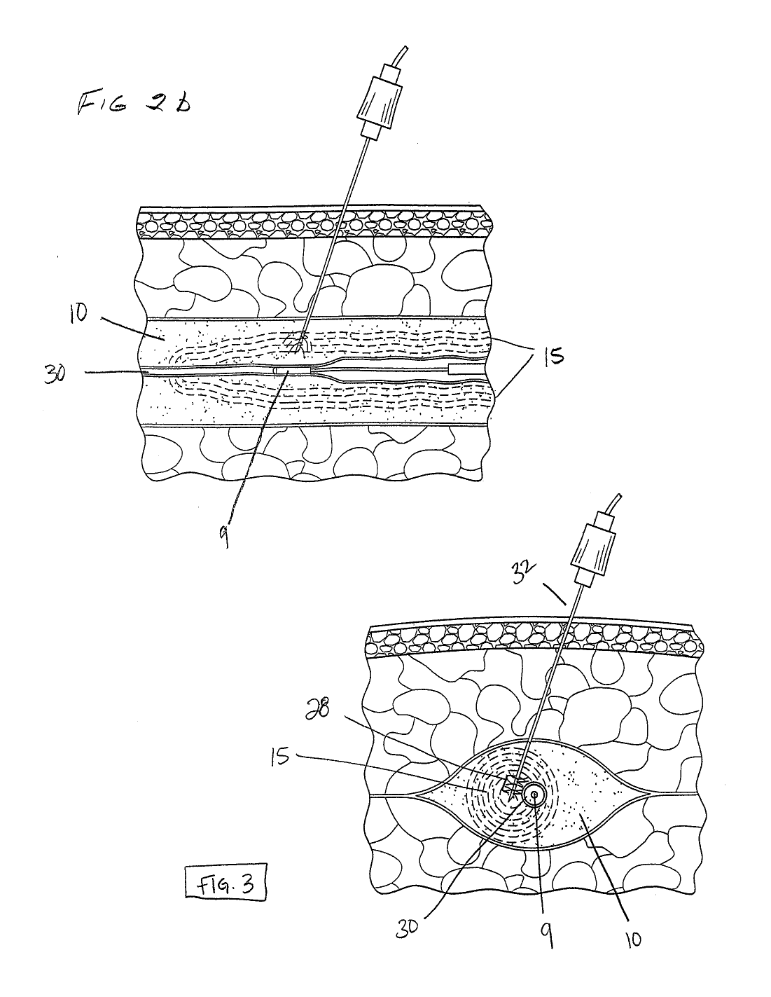

FIG. 5 shows an alternative embodiment of a needle 32, including a proximal end 14, a distal end 12, a pointed tip 16, and a hollow shaft 20, wherein the shaft body 18 contains solely multiple openings 28 along its length.

In a further embodiment, FIG. 6 illustrates a needle 32, with reflective scores surrounding each opening 40 along the shaft 18.

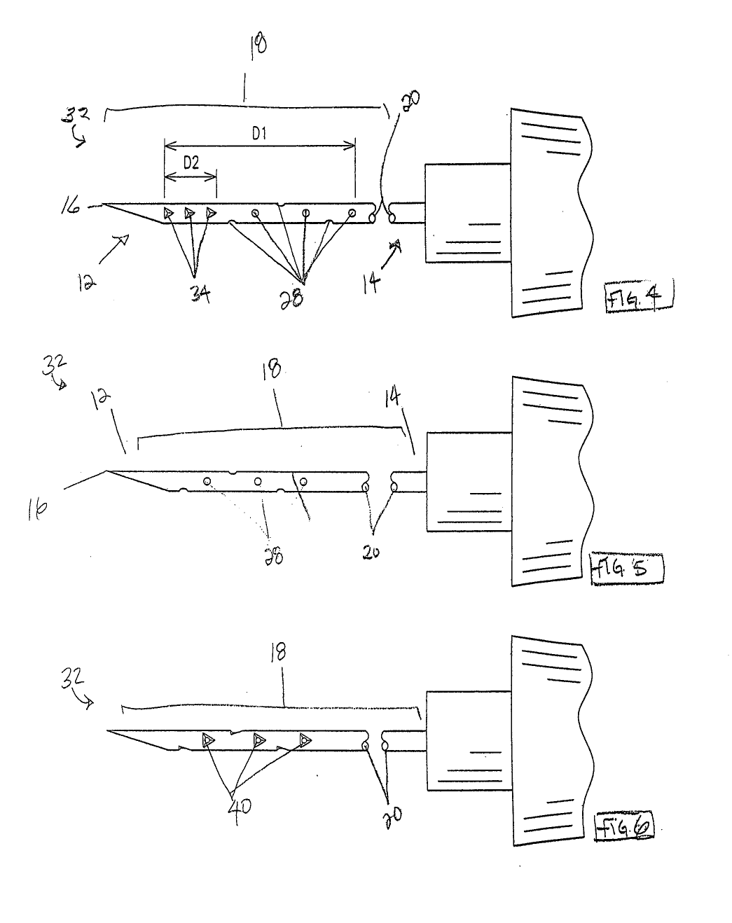

FIG. 7 shows an exemplary embodiment in which a needle 32 contains reflective scores surrounding each opening 42 along its shaft 18, wherein the diameter of each opening progressively increases from proximal end 14 to distal end 12 of the needle 32. The variation in diameter of the openings ranging from largest diameter at the distal end 12 of the needle to the smallest diameter at the proximal end 14 of the needle change the pressure of the fluid inside the needle while in use so that the fluid can more easily exit through the openings 42 as it travels down the length of the shaft 18 toward the distal end 12 of the needle 32.

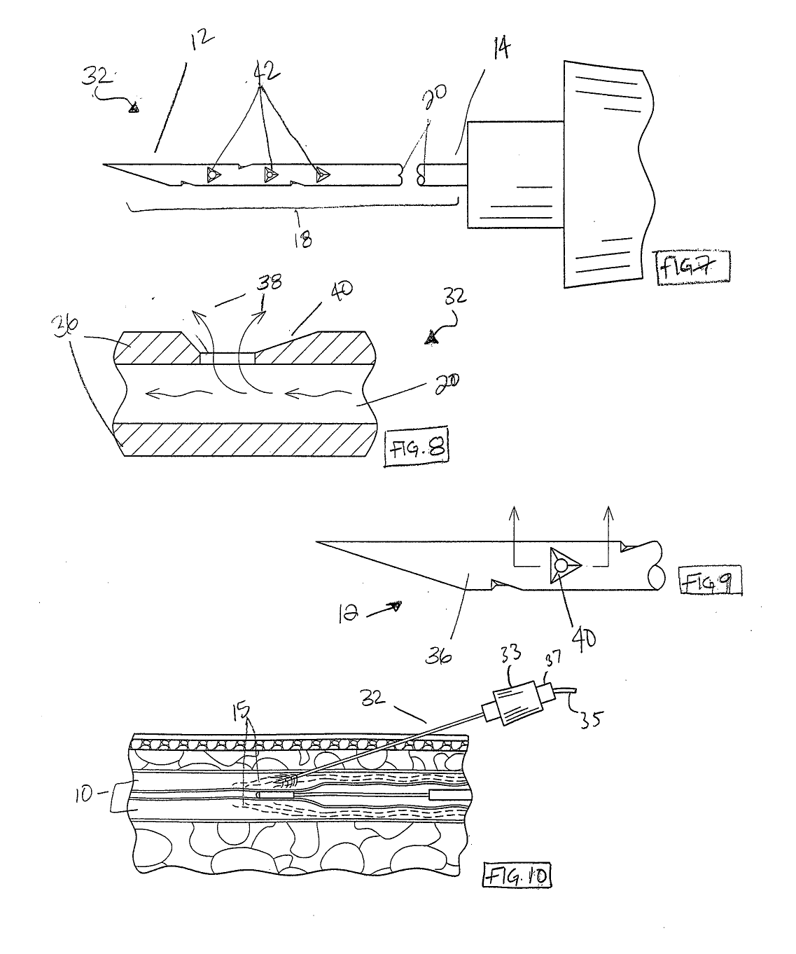

FIG. 8 is a cross sectional view of the wall 36 of a needle 32, according to the cross-sectional plane shown in FIG. 9 (see arrows) demonstrating the flow of tumescent anesthetic fluid 38 in the hollow center of the shaft 20, and through an opening which is surrounded by a reflective score 40 located in the wall of the needle 36.

FIG. 9 shows a side view of the distal portion of a needle 12, with reflective scores surrounding openings 40 in the needle wall 36, and arrows to demonstrate the axis upon which the cross section in FIG. 8 was taken.

FIG. 10 shows a more oblique angled approach, demonstrating the needle 32 providing a broad area of delivery of tumescent anesthetic fluid 15 to the area surrounding a target vein 10 via a single needle prick.

Shown in FIGS. 1 and 10, for example, is a base portion 33 contiguous to the proximal end 14. The base portion 33 assists the physician in manipulating the needle 32. Furthermore, it serves as juncture for engaging a catheter component 35. The catheter 35 delivers the tumescent anesthesia to the needle 32. The catheter can be provided with an end adapter 37 that removable engages to the base portion 33. One example of such engaging arrangement is a luer-lok type adapter/receiver.

Those skilled in the art will appreciate that the needle embodiments described herein can be made out of conventional materials according to specialized manufacturing methods. Materials include metal, glass, or polymers, or a combination thereof. The various embodiments described herein are for illustrative purposes only and that various modifications or changes in light thereof will be suggested to persons skilled in the art and are to be included within the spirit and purview of this application.

1. A method of delivering a tumescent anesthetic fluid to an area surrounding a target vein, comprising:
inserting a needle into a location proximate to the target vein;
said needle comprising a proximal end and a distal end, a pointed needle tip on said distal end; an elongated shaft body consisting of a hollow center, the hollow center of said shaft body extending throughout the entire length of said shaft body; said shaft body of a length sufficient to penetrate epidermal, dermal and subdermal layers of a patient's lower extremity; and multiple openings positioned along the length of said shaft body for administering the anesthetic through said openings into said area surrounding the target vein;
administering the tumescent anesthetic fluid to said location such that the tumescent anesthetic fluid ejects through said multiple openings simultaneously into the area surrounding the target vein.

2. The method of claim 1 wherein two or more reflective scores are positioned along the length of said shaft body.

3. The method of claim 2, wherein said scores are contiguous with said openings along said shaft body.

4. The method of claim 1 wherein said openings on said shaft body vary in diameter, and at least one opening has a different diameter than another opening.

5. The method of claim 1 wherein the opening at the tip of the needle has a smaller diameter than the openings along said shaft body.

6. A needle for injection of an anesthetic fluid, comprising:
a proximal end and a distal end, a pointed tip, and a hollow shaft;
multiple reflective scores positioned along said shaft; and
multiple openings positioned along said shaft.

7. The needle of claim 6, wherein said openings and scores are contiguous with one another along the length of said shaft.

8. The needle of claim 6, wherein the reflective scores surround each opening.

9. The needle of claim 6, wherein said multiple openings are positioned on said shaft beginning at a location 0.1 cm-1.5 cm from said distal end and ending at a location about 0.5 cm-5 cm from said distal end, wherein each opening is optionally spaced about 0.01-1 cm apart, and each opening is optionally 0.01-0.5 centimeters in diameter.

10. The needle of claim 9, wherein said openings are spaced equidistance apart.

11. (canceled)

12. (canceled)

13. (canceled)

14. A needle for injecting an area surrounding a target vein in a lower extremity of a patient, comprising:
a proximal end and a distal end, a hollow shaft connecting both ends, said distal end including a pointed tip, said pointed tip including a hole;
said needle length varies from 4-8 centimeters between said proximal and said distal ends;
said shaft contains a plurality of openings positioned along its length beginning at a location 0.1 cm-1.5 cm from said distal end and ending at a location 0.5 cm-5 cm from said distal end, wherein each opening is optionally spaced about 0.01-1 cm apart, and each opening is optionally 0.01-0.5 centimeters in diameter.

15. The needle of claim 14, wherein said shaft contains reflective scores positioned along its length.

16. The needle of claim 15, wherein said shaft contains reflective scores contiguous with each opening along the length of said shaft.

17. (canceled)
